(19) United States
(12) Patent Application Publication
(10) Pub. No.: US 2015/0080778 A1
(43) Pub. Date: Mar. 19, 2015

(54) BACK POSTURE STRAIGHTENING DEVICE

(71) Applicant: Paul CHEN, Vancouver (CA)

(72) Inventor: Paul CHEN, Vancouver (CA)

(73) Assignee: Paul CHEN, Vancouver (CA)

(21) Appl. No.: 14/028,704

(22) Filed: Sep. 17, 2013

(51) Int. Cl. A61F 5/02

(52) U.S. Cl. CPC A61F 5/026
USPC 602/19

(57) ABSTRACT

A back posture straightening device includes an elastic cable having two end portions secured together to form an endless loop, and a retainer bridge engaged between two segments of the elastic cable for separating the segments of the elastic cable from each other and for forming an eight (8)-shaped structure for the back posture straightening device and for engaging onto shoulder portions of a user, the retainer bridge includes a base element and an adjustable element engaged with the segments of the elastic cable, and the base element and the adjustable element are adjustable relative to each other for adjusting a distance between the segments of the elastic cable.

### BACKGROUND OF THE INVENTION

1. Field of the Invention

The present invention relates to an elastic exercise device or back posture training or exercising or straightening device, and more particularly to a back posture training or exercising or straightening device including an elastic rope or wire or cable supported with a retainer bridge to form an eight (8)-shaped structure or configuration for engaging with or onto the back or shoulder portion of the user and for training or exercising or straightening the shoulder portion and/or the back portion of the user.

2. Description of the Prior Art

Various kinds of typical elastic exercise devices have been developed and provided for conducting or operating various exercise operations and comprise an elastic rope or wire or cable for being operated to exercise various portions or various muscle groups of the user.

For example, U.S. Pat. No. 4,090,706 to Reda, U.S. Pat. No. 4,609,191 to Remme, U.S. Pat. No. 4,733,862 to Miller, U.S. Pat. No. 4,756,527 to Ledbetter, U.S. Pat. No. 4,909,505 to Tee, U.S. Pat. No. 5,318,494 to Santighian, U.S. Pat. No. 5,746,687 to Vial et al., U.S. Pat. No. 5,885,196 to Gvoich, U.S. Pat. No. 6,203,476 to Wang et al., U.S. Pat. No. 6,648,804 to Chen, and U.S. Pat. No. 6,866,617 to Chen disclose several of the typical elastic exercise devices each also comprising an elastic rope or wire or cable for being operated to exercise various portions or various muscle groups of the user.

However, the typical elastic exercise devices may only be pulled or operated or actuated to train or exercise the upper or lower muscle groups, but may not be used to train or exercise or straighten the shoulder portion and/or the back portion of the user.

U.S. Pat. No. 4,852,874 to Sleichter, III et al. disclose another typical elastic exercise device also comprising an elastic rope or wire or cable supported with a retainer ring or sleeve for forming an endless loop or for forming an eight (8)-shaped structure or configuration and for being operated to exercise various portions or various muscle groups of the user, and for training or exercising the upper or lower muscle groups of the user.

However, the typical elastic exercise devices may only be used or pulled or operated or actuated to train or exercise the upper or lower muscle groups, but may not be used to train or exercise or straighten the shoulder portion and/or the back portion of the user.

The present invention has arisen to mitigate and/or obviate the afore-described disadvantages of the conventional elastic exercise devices.

### SUMMARY OF THE INVENTION

The primary objective of the present invention is to provide a back posture straightening device including an elastic rope or wire or cable supported with a retainer bridge for forming an endless loop or for forming an eight (8)-shaped structure or configuration and for training or exercising or straightening the shoulder portion and/or the back portion of the user.

The other objective of the present invention is to provide a back posture straightening device including an elastic rope or wire or cable having two ends adjustably connected together with a coupler for applying an adjustable or changeable resilience or resilient force onto the user, and for allowing the back posture straightening device to be suitably and selectively engaged onto or with the shoulder portions of the users of different sizes or dimensions or standards.

In accordance with one aspect of the invention, there is provided a back posture straightening device comprising an elastic cable including a first end portion and a second end portion secured together to form an endless loop, and including an intermediate portion separated into a first segment and a second segment, and a retainer bridge engaged with the elastic cable and engaged between the first and the second segments of the elastic cable for separating the first and the second segments of the elastic cable from each other and for forming an eight (8)-shaped structure for the back posture straightening device and for engaging onto shoulder portions of a user, the retainer bridge includes a base element and an adjustable element engaged with the first and the second segments of the elastic cable respectively, and the base element and the adjustable element being adjustable relative to each other for adjusting a distance between the first and the second segments of the elastic cable.

The retainer bridge includes a holder member provided on the adjustable element for engaging with the first segment of the elastic cable. The holder member is preferably perpendicular to the adjustable element. The holder member includes at least one loop for slidably receiving and engaging with the first segment of the elastic cable.

The retainer bridge includes an anchoring member provided on the base element for engaging with the second segment of the elastic cable. The anchoring member includes a plate and a bulge extended from the plate for forming and defining a curved channel in the anchoring member and for slidably receiving and engaging with the second segment of the elastic cable.

The anchoring member includes a cover attached to the plate for engaging with the plate and the bulge and for anchoring the second segment of the elastic cable to the anchoring member of the base element.

The adjustable element includes at least one orifice formed therein, and the base element includes at least one projection extended therefrom for selectively engaging with the orifice of the adjustable element.

A coupler may further be provided and secured to the first end portion of the elastic cable, and the second end portion of the elastic cable is adjustable relative to the coupler. The coupler includes at least one lock ring provided thereon for selectively engaging with the second end portion of the elastic cable. The coupler includes a loop provided thereon for selectively engaging with the second end portion of the elastic cable.

A cushioning member may further be provided and attached to the intermediate portion of the elastic cable for softly and comfortably engaging with the user. The cushioning member includes at least one loop for selectively engaging with the intermediate portion of the elastic cable.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a rear plan schematic view illustrating the operation of a back posture straightening device in accordance with the present invention;

FIG. 2 is a partial front perspective view illustrating the operation of the back posture straightening device;

FIG. 3 is a partial exploded view of the back posture straightening device;

FIG. 4 is a perspective view of the back posture straightening device;

FIG. 5 is a front plan schematic view of the back posture straightening device; and

FIG. 6 is another front plan schematic view similar to FIG. 5, illustrating the operation of the back posture straightening device.

### DETAILED DESCRIPTION OF THE PREFERRED EMBODIMENT

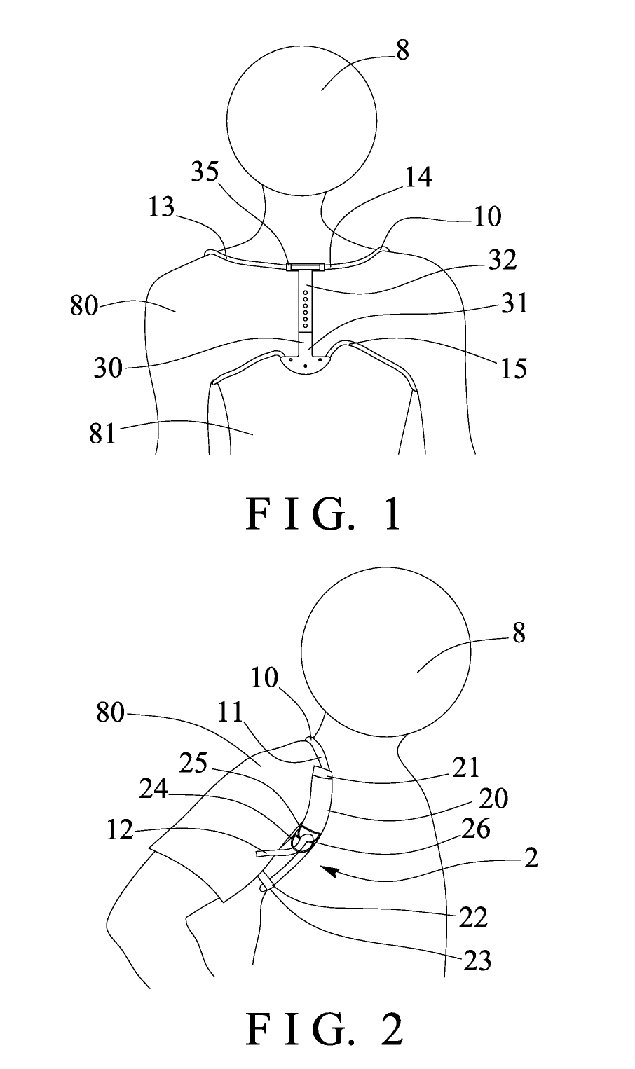

Referring to the drawings, and initially to FIGS. 1 and 2, a back posture straightening device in accordance with the present invention comprises an elastic rope or wire or cable 10 having two ends 11, 12 to be coupled or connected together with a coupler 2 for forming an endless loop and having a middle or intermediate portion 13 coupled or connected together with a retainer bridge 3 for forming an eight (8)-shaped structure or configuration and for engaging with or onto the shoulder portions 80 of a user 8 (FIGS. 1, 2), and for training or exercising or straightening the shoulder or back portion 81 of the user 8.

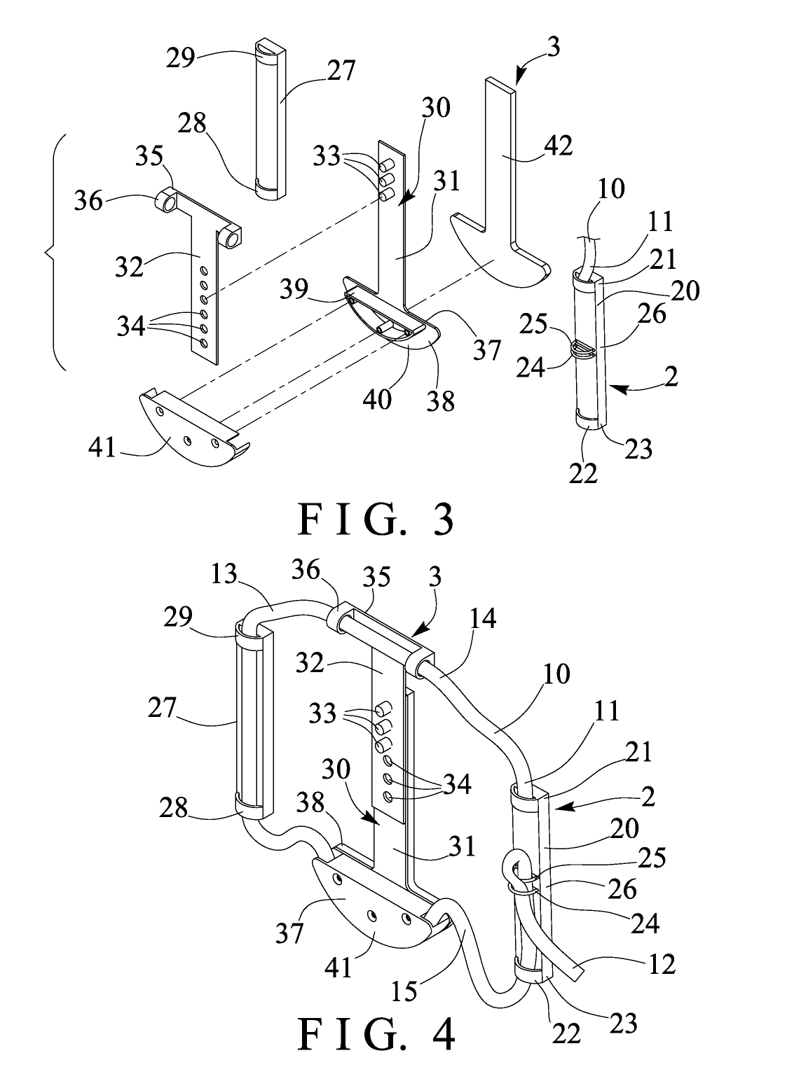

For example, as shown in FIGS. 3-6, the coupler 2 includes a longitudinal pad or cushioning member 20 made of soft or spongy or resilient materials for softly and comfortably engaging with or onto the shoulder portion 80 of the user 8, the one or first end portion 11 of the elastic cable 10 is solidly and stably attached or mounted or secured or engaged with the one or first end portion 21 of the cushioning member 20 with molding or hot-pressing procedures, adhesives, screws or bolts or catches or latches or fasteners or the like (not illustrated), and the cushioning member 20 includes a ring or hook or loop 22 attached or mounted or secured or provided on the other or second end portion 23 of the cushioning member 20 for threading or slidably receiving or engaging with or mounting or securing or attaching the other or second end portion 12 of the elastic cable 10.

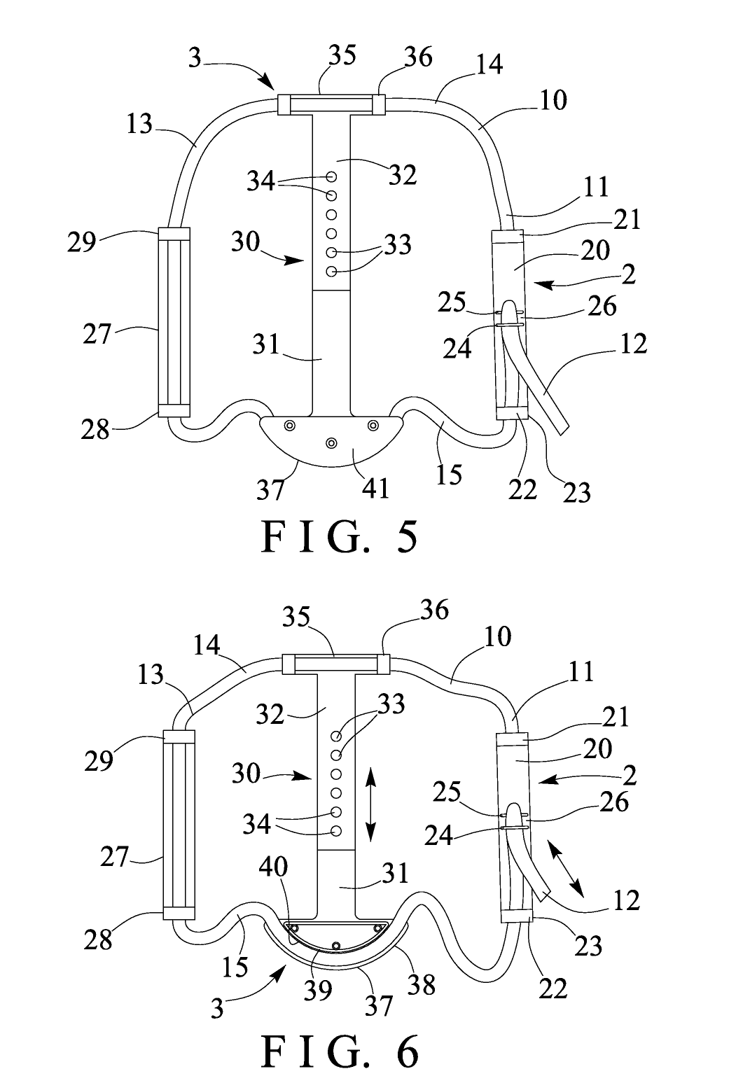

The cushioning member 20 further includes one or more (such as two) catch or latch or lock members or rings 24, 25 attached or mounted or secured or provided on the middle or intermediate portion 26 of the cushioning member 20 for engaging with or mounting or anchoring or retaining or positioning or attaching or securing or latching or locking the other or second end portion 12 of the elastic cable 10 to the cushioning member 20 and thus for forming an endless loop or structure or configuration for the elastic cable 10. As shown in FIG. 5, the other or second end portion 12 of the elastic cable 10 is engageable with the loop 22 at the other or second end portion 23 of the cushioning member 20 and/or engaged through the lock rings 24, 25, and may further be folded and engaged through one of the lock rings 24 for being solidly and stably secured or locked to the other or second end portion 23 of the cushioning member 20.

Another longitudinal pad or cushioning member 27 is also made of soft or spongy or resilient materials for softly and comfortably engaging with or onto the shoulder portion 80 of the user 8 and includes one or more (such as two) rings or hooks or loops 28, 29 attached or mounted or secured or provided thereon for slidably receiving or threading or engaging with the middle or intermediate portion 13 of the elastic cable 10 and for attaching or mounting or securing or retaining the cushioning member 27 to the middle or intermediate portion 13 of the elastic cable 10. The middle or intermediate portion 13 of the elastic cable 10 may be spaced or separated by the cushioning members 20, 27 into two portions or segments 14, 15, such as an upper or first portion or segment 14 and a lower or second portion or segment 15.

The retainer bridge 3 is threaded or engaged with or onto the middle or intermediate portion 13 of the elastic cable 10 and disposed or located between the cushioning members 20, 27 and/or between the segments 14, 15 of the elastic cable 10 for forming an eight (8)-shaped structure or configuration for the back posture straightening device and thus for allowing the back posture straightening device to be attached or mounted or secured or engaged with or onto the shoulder portion 80 and/or the back portion 81 of the user 8. The retainer bridge 3 includes a central support or stay member 30 having a lower base element 31 and an upper adjustable or movable element 32 adjustably attached or mounted or secured together or engaged with each other for extending or spacing or retaining or supporting or disengaging or separating the segments 14, 15 of the elastic cable 10 from each other for thus forming the eight (8)-shaped structure or configuration for the back posture straightening device.

For example, the lower base element 31 of the stay member 30 includes one or more (such as three) pegs or keys or tongues or projections 33 extended therefrom, and the upper adjustable or movable element 32 of the stay member 30 includes one or more (such as six) cavities or holes or orifices 34 formed therein for selectively receiving or engaging with the projections 33 of the lower base element 31 of the stay member 30 and for allowing the base element 31 and the adjustable element 32 of the stay member 30 to be moved or adjusted relative to each other. The stay member 30 includes a carrier or holder member 35 formed or provided on the adjustable element 32 and substantially perpendicular to the adjustable element 32 and having one or more (such as two) rings or hooks or loops 36 attached or mounted or secured or provided on the holder member 35 for slidably receiving or threading or engaging with the upper or first portion or segment 14 of the middle or intermediate portion 13 of the elastic cable 10.

The stay member 30 further includes a carrier or retainer or anchoring member 37 formed or provided on the base element 31 and having a panel or board or plate 38 and a projection or protrusion or swelling or bulge 39 extended from the plate 38 for forming or defining a curved passage or pathway or groove or slot or channel 40 in the anchoring member 37 and/or between the plate 38 and the bulge 39 and for slidably receiving or threading or engaging with the lower or second portion or segment 15 of the middle or intermediate portion 13 of the elastic cable 10, and further includes a cover 41 attached or mounted or secured to the plate 38 for engaging with the plate 38 and/or the bulge 39 and for solidly and stably anchoring or retaining or positioning the second segment 15 of the middle or intermediate portion 13 of the elastic cable 10 to the anchoring member 37 of the base element 31. A panel 42 includes a shape identical to or corresponding to that of the base element 31 and/or the plate 38 of the stay member 30 and attached or mounted or secured to the base element 31 and/or the plate 38 for reinforcing the same.

In operation, as shown in FIGS. 1 and 2, the back posture straightening device includes an eight (8)-shaped structure or configuration for selectively engaging with or onto the shoulder portions 80 of the user 8, and for training or exercising or straightening the shoulder portion 80 and/or the back portion 81 of the user 8. As shown in FIGS. 5 and 6, the adjustable element 32 of the stay member 30 is movable or adjustable relative to the base element 31 for adjusting the distance between the segments 14, 15 of the middle or intermediate portion 13 of the elastic cable 10, and in order to adjust the resilience or spring biasing force of the elastic cable 10 to the user 8, and/or for allowing the back posture straightening device to be suitably and selectively engaged onto or with the shoulder portions 80 and/or the back portion 81 of the users 8 of different sizes or dimensions or standards.

As shown in FIGS. 2 and 6, the other or second end portion 12 of the elastic cable 10 is further movable or adjustable relative to the cushioning member 20 of the coupler 2, particularly relative to the lock rings 24, 25 of the cushioning member 20 for adjusting the length of the endless loop of the elastic cable 10 or the eight (8)-shaped structure or configuration of the back posture straightening device and thus for allowing the back posture straightening device to be suitably and selectively engaged onto or with the shoulder portions 80 and/or the back portion 81 of the users 8 of different sizes or dimensions or standards.

Accordingly, the back posture straightening device in accordance with the present invention includes an elastic rope or wire or cable having two end portions coupled together for forming an endless loop and having a middle or intermediate portion supported with a retainer bridge for forming an eight (8)-shaped structure or configuration and for training or exercising or straightening the back portion of the user, the ends of the elastic rope or wire or cable are adjustably connected together with a coupler for applying an adjustable or changeable resilience or resilient force onto the user.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A back posture straightening device comprising:
an elastic cable including a first end portion and a second end portion secured together to form an endless loop, and including an intermediate portion separated into a first segment and a second segment, and
a retainer bridge engaged with said elastic cable and engaged between said first and said second segments of said elastic cable for separating said first and said second segments of said elastic cable from each other and for forming an eight (8)-shaped structure for said back posture straightening device and for engaging onto shoulder portions of a user, wherein
said retainer bridge includes a base element and an adjustable element engaged with said first and said second segments of said elastic cable respectively, and said base element and said adjustable element being adjustable relative to each other for adjusting a distance between said first and said second segments of said elastic cable.

2. The back posture straightening device as claimed in claim 1, wherein said retainer bridge includes a holder member provided on said adjustable element for engaging with said first segment of said elastic cable.

3. The back posture straightening device as claimed in claim 2, wherein said holder member is perpendicular to said adjustable element.

4. The back posture straightening device as claimed in claim 3, wherein said holder member includes at least one loop for slidably receiving and engaging with said first segment of said elastic cable.

5. The back posture straightening device as claimed in claim 1, wherein said retainer bridge includes an anchoring member provided on said base element for engaging with said second segment of said elastic cable.

6. The back posture straightening device as claimed in claim 5, wherein said anchoring member includes a plate and a bulge extended from said plate for forming and defining a curved channel in said anchoring member and for slidably receiving and engaging with said second segment of said elastic cable.

7. The back posture straightening device as claimed in claim 6, wherein said anchoring member includes a cover attached to said plate for engaging with said plate and said bulge and for anchoring said second segment of said elastic cable to said anchoring member of said base element.

8. The back posture straightening device as claimed in claim 1, wherein said adjustable element includes at least one orifice formed therein, and said base element includes at least one projection extended therefrom for selectively engaging with said at least one orifice of said adjustable element.

9. The back posture straightening device as claimed in claim 1 further comprising a coupler secured to said first end portion of said elastic cable, and said second end portion of said elastic cable is adjustable relative to said coupler.

10. The back posture straightening device as claimed in claim 9, wherein said coupler includes at least one lock ring provided thereon for selectively engaging with said second end portion of said elastic cable.

11. The back posture straightening device as claimed in claim 9, wherein said coupler includes a loop provided thereon for selectively engaging with said second end portion of said elastic cable.

12. The back posture straightening device as claimed in claim 1 further comprising a cushioning member attached to said intermediate portion of said elastic cable.

13. The back posture straightening device as claimed in claim 12, wherein said cushioning member includes at least one loop for selectively engaging with said intermediate portion of said elastic cable.

14. A back posture straightening device comprising:
an elastic cable including a first end portion and a second end portion to form an endless loop, and including an intermediate portion separated into a first segment and a second segment, and
a retainer bridge engaged with said elastic cable and engaged between said first and said second segments of said elastic cable for separating said first and said second segments of said elastic cable from each other and for forming an eight (8)-shaped structure for said back posture straightening device and for engaging onto shoulder portions of a user, wherein a coupler is secured to said first end portion of said elastic cable, and said second end portion of said elastic cable is adjustable relative to said coupler.

15. The back posture straightening device as claimed in claim 14, wherein said coupler includes at least one lock ring provided thereon for selectively engaging with said second end portion of said elastic cable.

16. The back posture straightening device as claimed in claim 14, wherein said coupler includes a loop provided thereon for selectively engaging with said second end portion of said elastic cable.

17. The back posture straightening device as claimed in claim 14, wherein said retainer bridge includes a base element and an adjustable element engaged with said first and said second segments of said elastic cable respectively, and said base element and said adjustable element are adjustable relative to each other for adjusting a distance between said first and said second segments of said elastic cable.

18. The back posture straightening device as claimed in claim 17, wherein said retainer bridge includes a holder member provided on said adjustable element for engaging with said first segment of said elastic cable.

19. The back posture straightening device as claimed in claim 17, wherein said retainer bridge includes an anchoring member provided on said base element for engaging with said second segment of said elastic cable.

20. The back posture straightening device as claimed in claim 17, wherein said adjustable element includes at least one orifice formed therein, and said base element includes at least one projection extended therefrom for selectively engaging with said at least one orifice of said adjustable element.
